(19) United States
(12) Patent Application Publication
(10) Pub. No.: US 2016/0249972 A1
(43) Pub. Date: Sep. 1, 2016

(54) ELECTROSURGERY ARRANGEMENT, GUIDE SLEEVE AND METHOD FOR OPERATING AN ELECTROSURGERY ARRANGEMENT

(71) Applicant: OLYMPUS WINTER & IBE GMBH, Hamburg (DE)

(72) Inventor: German KLINK, Berlin (DE)

(73) Assignee: OLYMPUS WINTER & IBE GMBH, Hamburg (DE)

(21) Appl. No.: 15/026,775

(22) PCT Filed: Jul. 31, 2014
(86) PCT No.: PCT/EP2014/066507

§ 371 Date: Apr. 1, 2016

(30) Foreign Application Priority Data
Oct. 1, 2013 (DE) 10 2013 219 927.9

(51) Int. Cl. A61B 18/14

(52) U.S. Cl. CPC A61B 18/14; A61B 2018/1472; A61B 2018/00541

(57) ABSTRACT

An electrosurgery arrangement, including: an electrosurgical probe having a rod-shaped probe body, an outer surface and also one distal and one proximal electrode, wherein the distal electrode and the proximal electrode each form an electrically conductive section of the outer surface and are electrically insulated from one another, and wherein an expandable, electrically insulating probe separator is arranged between the distal electrode and the proximal electrode; and a guide sleeve having a lumen into which the electrosurgical probe is removably introduced and having an expandable, electrically insulating sleeve separator, wherein the guide sleeve has at least one respective radial opening both proximally and distally relative to the sleeve separator, and a method for operating such an electrosurgery arrangement.

The invention relates to an electrosurgery arrangement comprising an electrosurgical probe with a rod-shaped probe body, an outer surface, and also a distal electrode and a proximal electrode, wherein the distal electrode and the proximal electrode each form an electrically conductive portion of the outer surface and are electrically insulated from each other, and wherein an expandable, electrically insulating probe separator is arranged between the distal electrode and the proximal electrode.

The invention further relates to a guide sleeve, and to a method for operating an electrosurgery arrangement and the use of an electrosurgical probe.

Electrosurgery arrangements with electrosurgical probes having two electrodes, so-called bipolar probes, are used to treat body tissue. The two electrodes are connected to poles of an RF generator in order to deliberately ablate, coagulate or cut body tissue, for example a tumor. For this purpose, the electrosurgical probe, with the two insulated electrodes between which the RF voltage is applied, is guided directly to the target location in the body, and the current circuit is closed via the tissue lying between the electrodes, which tissue is thereby ablated, coagulated or cut.

Electrosurgical probes with a rod-shaped probe body, which has a longitudinal direction, are used in particular for insertion into lumina of the body, for example the lungs. To enlarge the electrodes, for example in order to establish contact with the walls of a body lumen, for example of a bronchus, and thereby be able to treat a target location, for example a tumor, adjoining the body lumen, an electrically conductive fluid can be used. The latter is introduced into the body lumen, for example, and is there in contact with the electrodes formed on the outer surface of the rod-shaped probe body. To be able to maintain the electrical insulation of the two electrodes of the probe body even in the electrically conductive fluid, for example an electrically conductive gel, preferably a 20% NaCl gel, the document DE 10 2011 085 616.1 discloses an electrosurgical probe with an expanding separation element, which separates the gel inside the body lumen into two mutually insulated areas, such that a current introduced into the gel via the probe electrodes has to flow through tissue delimiting the body lumen. For the delivery of the gel or electrically conductive fluid, the solution disclosed in DE 10 2011 085 616.1 provides an opening in the probe body. In practice, however, it has been found that further improvements are desirable.

It is therefore an object of the present invention to make available an electrosurgery arrangement and a method for operating an electrosurgery arrangement, which provide an alternative solution to already existing solutions.

In particular, it is an object of the present invention to make available an electrosurgery arrangement and a method for operating an electrosurgery arrangement, which are easier to manage and more reliable to use compared to existing solutions.

According to the invention, this object is achieved by an electrosurgery arrangement comprising an electrosurgical probe with a rod-shaped probe body, an outer surface and also a distal electrode and a proximal electrode, wherein the distal electrode and the proximal electrode each form an electrically conductive portion of the outer surface and are electrically insulated from each other, and wherein an expandable, electrically insulating probe separator is arranged between the distal electrode and the proximal electrode; and a guide sleeve with a lumen into which the electrosurgical probe is removably inserted, and with an expandable, electrically insulating sleeve separator, wherein the guide sleeve has at least one radial opening both proximally and distally relative to the sleeve separator.

The invention is based among other things on the recognition that, when using existing electrosurgical probes with gel electrodes, it can happen that an ablation treatment concludes too early, for example after just 5 minutes, instead of the expected 30 minutes. The invention is based on the recognition that too early a conclusion of the ablation treatment may be the result of an uneven, incorrect or insufficient distribution of the gel. In such a case, the probe has to be withdrawn or at least moved in order to be able to meter in sufficient gel to the correct places. The probe then has to be correctly positioned again relative to the target location. This is a difficult maneuver and, moreover, it is time-consuming. Particularly in the case of tumor ablations, very precise positioning may be necessary, for example using CT fluoroscopy, and the latter would then also be required accordingly in the case of a repositioning after metering-in of further gel. By contrast, with the solution according to the invention, these disadvantages are avoided or greatly reduced.

The electrosurgery arrangement according to the invention comprises in the first instance an electrosurgical probe with a rod-shaped probe body. The probe body extends in a longitudinal direction and has a mostly cylindrical outer surface. A portion of this outer surface forms a distal electrode, while a further portion forms a proximal electrode. The two electrodes are electrically insulated from each other. An expandable, electrically insulating probe separator is arranged between the two electrodes namely the distal electrode and the proximal electrode. This probe separator is expandable substantially orthogonally with respect to the longitudinal direction of the probe body.

Moreover, the electrosurgery arrangement has a guide sleeve with a lumen into which the electrosurgical probe is removably inserted. The guide sleeve can also be designated as a guide tube or guide catheter, is generally in the shape of a hose or hollow cylinder and generally has a jacket with an inner jacket surface and an outer jacket surface and a mostly annular cross section orthogonal to the longitudinal direction. The guide sleeve has an external diameter which is preferably chosen such that the guide sleeve is insertable into a body lumen, for example a bronchus, as far as a target location.

The electrosurgical probe is generally inserted into the guide sleeve from the direction of the proximal end. For this purpose, the electrosurgical probe has an external diameter which is adapted to an internal diameter of the guide sleeve in such a way that the electrosurgical probe can be inserted into the guide sleeve and removed again from the latter, i.e. the external diameter of the electrosurgical probe is smaller than the internal diameter of the guide sleeve.

The guide sleeve has an expandable, electrically insulating sleeve separator. The sleeve separator is expandable substantially orthogonally with respect to the longitudinal direction of the guide sleeve, which preferably coincides with the longitudinal direction of the probe body. Arranged in the proximal and distal directions from this sleeve separator there is in each case at least one radial opening. These radial openings in the jacket of the guide sleeve fluidically connect the lumen of the guide sleeve to an environment of the guide sleeve.

A radial opening is understood here as an opening in the jacket of the guide sleeve which establishes a lateral connection between the lumen of the guide sleeve and an environment of the guide sleeve. While the term “radial opening” in a narrower sense designates an opening oriented exactly orthogonally with respect to the longitudinal direction, i.e. at 90° to the longitudinal direction, radial is also used here to designate those openings that are arranged obliquely with respect to the longitudinal direction, for example at an angle of more than 10° and of less than 90° to the longitudinal direction.

The sleeve separator is designed such that, in an expanded state, it preferably touches the wall of a body lumen and separates the body lumen, transversely or substantially orthogonally with respect to the longitudinal direction, into two separate areas. The probe separator is preferably designed such that, in the expanded state, it touches an inner wall or inner jacket surface of the guide sleeve and separates the lumen of the guide sleeve into two separate areas substantially orthogonally with respect to the longitudinal direction.

The sleeve separator and the probe separator are both preferably convertible, in each case via an actuation mechanism, from a contracted state to an expanded state and vice versa, wherein the cross section orthogonal to the longitudinal direction of the sleeve separator and of the probe separator is greater in their respectively expanded states than it is in their respectively contracted states.

In the contracted state, the external diameter of the sleeve separator is preferably the same as or only slightly larger than the external diameter of the rest of the guide sleeve. Moreover, the external diameter of the probe separator in the contracted state is preferably the same as or only slightly larger than the external diameter of the rest of the electrosurgical probe. In the expanded state, the external diameter of the sleeve separator is preferably large enough to circumferentially contact the wall of a body lumen orthogonally with respect to the longitudinal direction, so as to ensure a reliable separation of the body lumen into two areas insulated from each other. Similarly, the external diameter of the probe separator in the expanded state is preferably so large that the inner wall of the guide sleeve is circumferentially contacted and the lumen of the guide sleeve is thus separated, substantially orthogonally with respect to the longitudinal direction, into two areas insulated from each other.

The electrosurgery arrangement according to the invention has various advantages: By virtue of the combination of a guide sleeve and of an electrosurgical probe guided therein, it is possible, at the start of a treatment, to place the guide sleeve exactly at the target location in the body lumen. By expansion of the sleeve separator, not only can the body lumen be separated into two separate areas, the guide sleeve can preferably also be anchored or fixed at the desired position. By virtue of the fact that the guide sleeve has at least one radial opening both distally and also proximally relative to the sleeve separator, it is possible for electrically conductive fluid delivered through the lumen of the guide sleeve to emerge on both sides of the sleeve separator, i.e. proximally and distally, even in the expanded state of the sleeve separator, and pass from the lumen of the guide sleeve through the radial openings into the body lumen and become distributed there.

In particular, electrically conductive fluid can be introduced in a particularly simple manner through the lumen of the guide sleeve and through the radial openings into the body lumen, and more can also be metered in during the treatment, since the electrosurgical probe is removable from the lumen and, thereafter, substantially the entire lumen of the guide sleeve is available tor introduction of the electrically conductive fluid. The introduction of electrically conductive fluid into the lumen of the guide sleeve can take place, for example, via a hose that is inserted into the lumen of the guide sleeve. The electrically conductive fluid used is preferably a gel which, among other things, has the advantage that, on account of its viscosity, the applied gel largely remains at the application site. However, the electrically conductive fluid used can also be a liquid with a viscosity lower than that of a gel, in which case measures are then preferably taken to ensure that sufficient liquid is present at the application site or at the target location. Embodiments described below with gel as the electrically conductive fluid can also be used with other electrically conductive fluids.

When sufficient gel has been introduced through the openings into the body lumen before or at the start of treatment, or when a required additional amount has been metered in during treatment, the electrosurgical probe can be inserted into the lumen of the guide sleeve. Since gel is likewise present in the lumen of the guide sleeve, expansion of the probe separator ensures that two separate areas are also obtained inside the lumen of the guide sleeve, so as to form two separate gel electrodes.

If it is necessary for more gel to be metered in during treatment, the probe separator can be contracted and removed from the lumen of the guide sleeve, such that the lumen of the guide sleeve is once again available for the introduction of gel, for example by means of a hose.

By contrast, the sleeve separator can remain expanded throughout the entire treatment and is thus able to maintain the exact position obtained at the start of treatment. This facilitates and improves the handling of the electrosurgery arrangement since, on the one hand, the exact positioning is maintained throughout the entire period of the treatment and, on the other hand, the time spent on repositioning is avoided, such that time is saved even if more gel has to be metered in during the treatment.

In a preferred embodiment of the electrosurgery arrangement, provision is made that the distal end of the guide sleeve is closed.

Here, a closed end is understood as meaning an end that is fully closed, but also an end that is partially closed and partially open, for example with a lattice structure or web structure. It is important that a distal end of an electrosurgical probe is prevented from passing through the closed end of the guide sleeve. Accordingly, closed is understood here as meaning that the distal end or the guide sleeve is closed to passage of the distal end of the electrosurgical probe, but passage of fluid through the closed end of the guide sleeve need not be excluded. A hose-shaped insertion catheter, for example, can have a closed and preferably rounded distal end.

Such a configuration with a closed distal end has the advantage that, upon insertion of the electrosurgical probe into the lumen of the guide sleeve, a kind of limit stop is forced by the closed distal end of the guide sleeve, that is to say the electrosurgical probe can be inserted with its distal end no farther than the distal end of the guide sleeve. In this way, after exact positioning of the guide sleeve and expansion of the sleeve separator, the electrosurgical probe can be repeatedly removed from the guide sleeve and inserted back into the guide sleeve and, as long as the distal end of the probe body is inserted as far as the distal end of the guide sleeve, the position of the electrosurgical probe, and in particular of its electrodes, can be made exactly reproducible.

It is particularly preferable that, with the electrosurgical probe inserted into the lumen as far as the distal end of the guide sleeve, the distal electrode is arranged inside the lumen in the area of the opening arranged distally relative to the sleeve separator, and the proximal electrode is arranged inside the lumen in the area of the opening arranged proximally relative to the sleeve separator.

In this arrangement, the guide sleeve with the radial openings and the electrosurgical probe with the electrodes are designed in such a way that the radial opening arranged proximally relative to the sleeve separator and the proximal electrode of the probe at least partially overlap in the longitudinal direction of the probe body when the probe is inserted into the lumen of the guide sleeve, with its distal end reaching as far as the distal end of the guide sleeve. It is likewise preferable that the radial opening arranged distally relative to the sleeve separator and the distal electrode of the probe at least partially overlap in the longitudinal direction of the probe body when the probe is inserted into the lumen of the guide sleeve, with its distal end reaching as far as the distal end of the guide sleeve. This means that in particular the distances of the proximal and distal electrodes from each other and from the distal end of the probe body and their respective extent in the longitudinal direction are preferably adapted to the radial openings arranged proximally and distally from the sleeve separator, and their distances from the distal end of the guide sleeve and their extent in the longitudinal direction are adapted to each other. In this way, it is possible to ensure that, when the guide sleeve is exactly positioned and is fixed in this position by the expanded sleeve separator, the probe body inserted into the lumen of the guide sleeve likewise repeatedly and reproducibly adopts the exact desired position, in particular in relation to the target location. When inserting the probe into the lumen of the guide sleeve, an operator can tell when the distal end of the probe body has reached the distal end of the guide sleeve and has thus arrived at the desired position. The adapted arrangement of the electrodes and of the respective radial openings ensures that, between the respective electrode, the gel located in the lumen of the guide sleeve and the gel located in the body lumen, it is possible for a continuous gel electrode to form from the probe electrode to the wall of the body lumen through the respective radial opening in the jacket of the guide sleeve.

Moreover, it is particularly preferable that the guide sleeve has several radial openings both proximally and also distally relative to the sleeve separator. The provision of several radial openings in the jacket of the guide sleeve, both proximally and also distally relative to the sleeve separator, is advantageous for ensuring sufficient distribution of the gel in the body lumen surrounding the guide sleeve.

In a particularly advantageous embodiment, provision is made that the several radial openings are each distributed, proximally and distally relative to the sleeve separator, in the circumferential direction of the guide sleeve, preferably uniformly distributed. It is thus possible to ensure that gel introduced into the lumen of the guide sleeve can emerge uniformly about the entire circumference of the guide sleeve in the area of the openings. Preferably, openings are provided that are distributed in a range of 360° orthogonally with respect to the longitudinal direction.

It is also preferable that the openings have the same size and/or the same shape. Alternatively, the openings can also have different sizes and shapes.

In an embodiment of the openings that is preferred in practice, the openings are in the shape of elongate lenses or broad slits which extend with their longest extent in the longitudinal direction of the guide sleeve. The length of the openings in the longitudinal direction preferably corresponds substantially to the length of the respective electrode in the longitudinal direction of the probe body.

In order to ensure or improve the formation of the gel electrode between the probe electrode and the wall of the body lumen, it is preferable for the guide sleeve to have openings with the largest possible surface area in order to improve the fluidic connection and in particular the electrical cross section between the gel-filled lumen of the guide sleeve and the gel-filled body lumen. At the same time, sufficient stability of the guide sleeve must be ensured. Further possibilities, either individually or in combination, for improving the formation of a gel electrode via the openings of the guide sleeve are set forth in the following preferred embodiments.

For example, it is preferable that electrically conductive surfaces connected to each other are formed between the openings arranged proximally relative to the sleeve separator. It is moreover preferable that electrically conductive surfaces connected to each other are formed between the openings arranged distally relative to the sleeve separator.

In another preferred embodiment, provision is made that the guide sleeve, in the area of the openings arranged proximally relative to the sleeve separator and/or in the area of the openings arranged distally relative to the sleeve separator, is made from an electrically conductive material or has an electrically conductive material.

Moreover, it may be preferable that the openings arranged proximally relative to the sleeve separator and/or the openings arranged distally relative to the sleeve separator are designed as a lattice structure, wherein the lattice structure is preferably made from an electrically conductive material or has an electrically conductive material.

In another preferred embodiment, provision is made that the guide sleeve has a lattice structure in the area of the openings arranged proximally relative to the sleeve separator and/or in the area of the openings arranged distally relative to the sleeve separator, wherein the lattice structure is preferably made from an electrically conductive material or has an electrically conductive material.

Moreover, provision can preferably be made that the edge of the openings arranged proximally relative to the sleeve separator and/or of the openings arranged distally relative to the sleeve separator is made completely or partially from an electrically conductive material or has an electrically conductive material.

In these various embodiments mentioned above, provision is made for electrically conductive material, for example metal, to be used in the area of the radial openings arranged in the guide sleeve proximally and/or distally from the sleeve separator. Such an embodiment with electrically conductive material, which can be provided for example as an electrically conductive coating of surfaces between the openings and/or at the edge of the openings and/or on a lattice structure, improves the formation of a continuous and coherent gel electrode on both sides of the openings, that is to say inward as seen from the guide sleeve, directed toward the lumen of the guide sleeve, and outward as seen from the guide sleeve, i.e. directed toward the body lumen. Since a direct fluidic connection is provided only through the openings themselves, while a direct connection of the gel is interrupted between the openings, it is preferable that the openings for forming the gel electrode have the largest possible surface area. At the same time, however, sufficient stability of the guide sleeve must be ensured so as to be able to safely insert it into a body lumen and remove it from same. Lattice structures, in particular metal lattices, can provide such stability while at the same time ensuring a large surface area of the openings. Particularly if the lattice is made from an electrically conductive material or has such a material, the formation of a gel electrode via the lattice or through the lattice is improved. If the openings are configured as continuous recesses through the jacket surface of the guide sleeve, it may be expedient to produce the guide sleeve in this area from an electrically conductive material or to coat it with such a material, in order to be able to use the jacket surface of the guide sleeve between the openings to support the formation of a continuous gel electrode.

In another preferred embodiment of the electrosurgery arrangement, provision is made that the expandable, electrically insulating sleeve separator and/or the expandable, electrically insulating probe separator are/is designed as an expandable balloon or as a deployable member.

The sleeve separator and/or the probe separator are preferably convertible, in each case via an actuation mechanism, from a contracted state to an expanded state and back again to the contracted state. The respective actuation mechanism can preferably be actuated or triggered from a proximal end of the electrosurgery arrangement.

The probe separator and/or the sleeve separator are in each case preferably formed circumferentially about the outer circumference of the guide sleeve and of the probe body, in order to achieve an effect of the probe separator or of the sleeve separator about the entire circumference of the guide sleeve and of the probe body. Preferably, the cross-sectional shape of the probe separator and/or of the sleeve separator is adapted to the respective cross-sectional shape of the guide sleeve or of the probe body, i.e., in the case of a probe body with a circular cross section, it is also preferred to have a circular or annular cross section of the probe separator, and, in the case of a guide sleeve with an annular cross section, it is preferable to have an annular cross section of the sleeve separator. Preferably, the probe separator and/or the sleeve separator have/has a design in which the cross-sectional shape is substantially the same or similar (for example annular) in the expanded state and in the contracted state, but the cross-sectional size changes, namely from the contracted to the expanded state (e.g. the ring in the expanded state has a greater diameter). A design of the sleeve separator and/or of the probe separator with a circular outer circumference in the expanded state is particularly preferred for use in hollow organs that likewise have a substantially circular cross section, for example in a bronchus.

The embodiments described below for separators can be used both tor the sleeve separator and also for the probe separator. The probe separator and the sleeve separator of an electrosurgery arrangement can be of an identical, similar or different configuration.

For example, a separator can be designed as an expandable envelope, which is formed by a portion of the outer surface of the probe body or of the guide sleeve. In this case, the separator can preferably be converted from the contracted to the expanded state by expansion of the envelope. For example, the expansion of the envelope can take place hydraulically or pneumatically by means of an actuation fluid, wherein a closed chamber is preferably formed in the separator, which chamber can be filled with the actuation fluid and permits controlled expansion.

Further possibilities for the design of a separator are, for example, an upsetting mechanism which, when actuated, causes a pushing-together in the longitudinal direction and thus brings about an expansion substantially orthogonally with respect to the longitudinal direction. A further possibility is a deploy able member with a fixed end and with a free, deployable end. Possible embodiments of separators are also described in particular in DE 10 2011 085 616.1 and can be used here. It is important that both the probe separator and also the sleeve separator achieve a separating effect adapted to the electrically conductive fluid to be used, i.e. it is important to ensure that, in the expanded state of the probe separator or of the sleeve separator, the electrically conductive fluid to be used cannot pass the separator.

According to a further aspect of the invention, the aforementioned object is achieved by a guide sleeve with a lumen into which an electrosurgical probe is removably insertable, and with an expandable, electrically insulating sleeve separator, wherein the guide sleeve has at least one radial opening proximally and distally relative to the sleeve separator. The guide sleeve is in particular designed for use in an electrosurgery arrangement as described above.

According to a further aspect of the invention, the aforementioned object is achieved by a method for operating an electrosurgery arrangement as described above, comprising the following steps: a guide sleeve with a lumen, into which an electrosurgical probe is removably insertable, and with an expandable, electrically insulating sleeve separator, wherein the guide sleeve has at least one radial opening both proximally and distally relative to the sleeve separator, is inserted into a body lumen as far as a target location, the sleeve separator is expanded, electrically conductive fluid is introduced into the lumen of the guide sleeve, an electrosurgical probe with a rod-shaped probe body, an outer surface and also a distal electrode and a proximal electrode, wherein the distal electrode and the proximal electrode each form an electrically conductive portion of the outer surface and are electrically insulated from each other, and wherein an expandable, electrically insulating probe separator is arranged between the distal electrode and the proximal electrode, is inserted into the lumen of the guide sleeve, the probe separator is expanded, and a bipolar RF voltage is applied to the electrodes.

The method is preferably extended by the following steps: removing the electrosurgical probe from the lumen of the guide sleeve, introducing electrically conductive fluid into the lumen of the guide sleeve again, inserting the electrosurgical probe into the lumen of the guide sleeve again, expanding the probe separator again, applying a bipolar RF voltage to the electrodes again.

According to a further aspect of the invention, the aforementioned object is achieved by the use of an electrosurgical probe with a rod-shaped probe body, an outer surface and also a distal electrode and a proximal electrode, wherein the distal electrode and the proximal electrode each form an electrically conductive portion of the outer surface and are electrically insulated from each other, and wherein an expandable, electrically insulating probe separator is arranged between the distal electrode and the proximal electrode, and/or a guide sleeve with a lumen into which an electrosurgical probe is removably insertable, and with an expandable, electrically insulating sleeve separator, wherein the guide sleeve has at least one radial opening both proximally and distally relative to the sleeve separator, in an electrosurgery arrangement as described above and/or in a method as described above for operating an electrosurgery arrangement.

Regarding the advantages, variants and design details of the aforementioned further aspects of the invention and their respective developments, reference is made to the above description of the corresponding features and developments of the electrosurgery arrangement.

A preferred embodiment of the invention is described by way of example and with reference to the attached figures, in which:

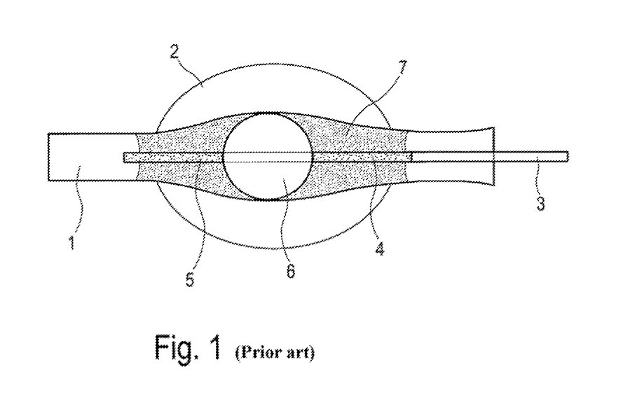

FIG. 1 shows a known electrosurgical probe for use with gel electrodes (prior art);

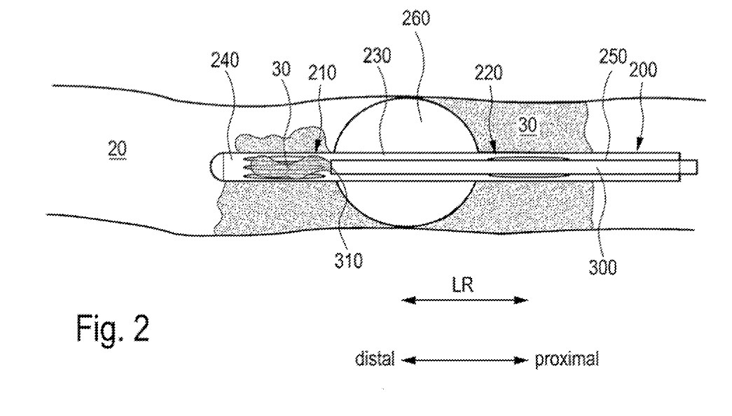

FIG. 2 shows an example of an electrosurgery arrangement during introduction of gel;

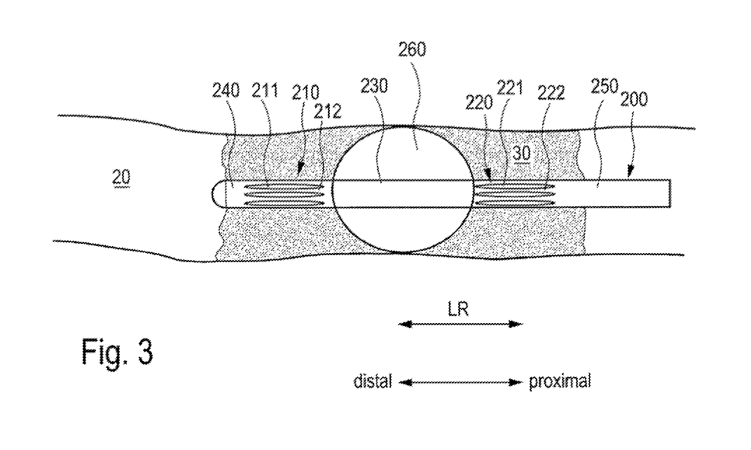

FIG. 3 shows the electrosurgery arrangement according to FIG. 2 with the gel introduced;

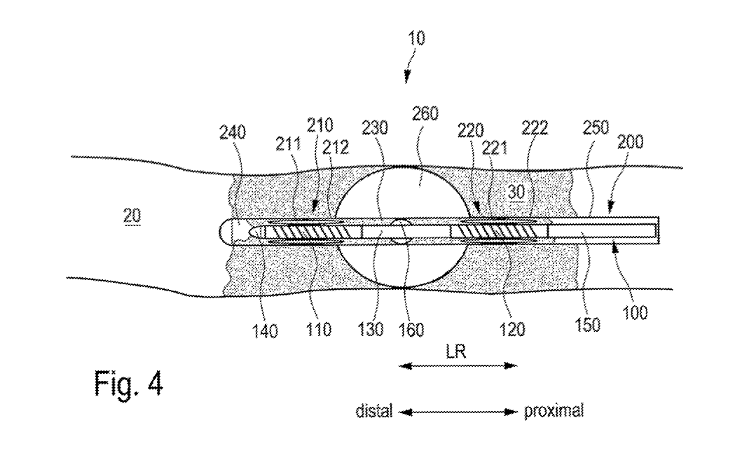

FIG. 4 shows the electrosurgery arrangement of FIGS. 2 and 3 with an inserted electrosurgical probe; and

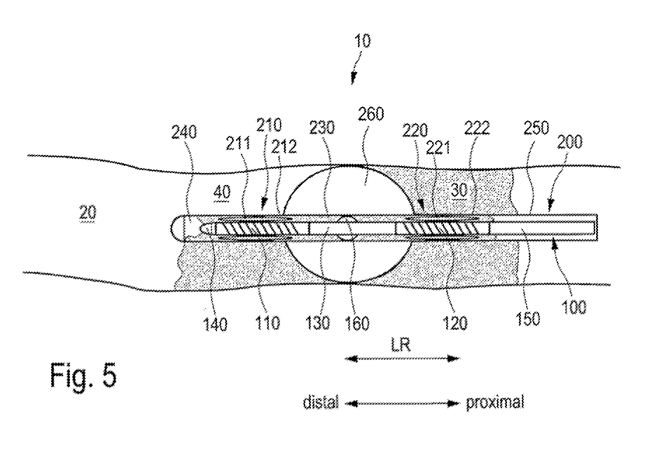

FIG. 5 shows the electrosurgery arrangement according to FIG. 4 with unevenly distributed gel.

The treatment of body tissue using electrosurgical probes with gel electrodes is known from the prior art. As can be seen from FIG. 1, an electrosurgical probe 3 with a distal electrode 5 and with a proximal electrode 4 is inserted into a bronchus 1 in the area of a target location 2, for example a tumor. An electrically insulating balloon 6, shown in the expanded state in FIG. 1, separates the body lumen of the bronchus 1 into two separate areas, such that the gel 7 is likewise separated into two areas and is in contact either with the proximal, electrode 4 or the distal electrode 5, such that an RF voltage applied to the electrodes leads to a flow of current through the body tissue in the area of the target location 2.

FIGS. 2-5 are schematic representations showing an example of an electrosurgery arrangement 10 according to the invention and its use. Elements that are identical or that have substantially the same function are provided with the same reference signs in FIGS. 2-5.

FIGS. 2 and 3 show only the guide sleeve 200 of the electrosurgery arrangement 10; FIGS. 4 and 5 also show the electrosurgical probe 100.

The electrosurgery arrangement 10 has a guide sleeve 200 with a lumen into which an electrosurgical probe 100 can be removably inserted from the direction of the proximal end. The guide sleeve 200 is designed as a hose-shaped guide catheter or guide tube with a jacket and an outer and an inner jacket surface. The guide sleeve 200 has a longitudinal direction LR and, orthogonally with respect to this longitudinal direction LR, a preferably annular cross section. The guide sleeve 200 has a closed, rounded distal end 240.

Moreover, the guide sleeve 200 has, in an area 230, an expandable and electrically insulating sleeve separator 260, which is shown in the expanded state in FIGS. 2-5. The sleeve separator 260 is designed as an expandable balloon, which is expandable substantially orthogonally with respect to the longitudinal direction LR, as is shown in FIGS. 2-5. The maximum external diameter of the sleeve separator 260 is preferably chosen such that a body lumen 20, for example a bronchus, is filled in cross section by the sleeve separator 260, so as to be able to ensure the separation of the body lumen 20 into two areas.

In an area 210 located distally from the sleeve separator 260, several radial openings 211 are provided in the jacket of the guide sleeve 200 and are distributed uniformly in the circumferential direction of the guide sleeve 200. In an area 220 located proximally relative to the sleeve separator 260, several radial openings 221 are likewise provided, and these likewise are distributed uniformly in the circumferential direction of the guide sleeve 200. Between the radial openings 211 and 221, which are each designed as elongate, lens-shaped openings, there are areas 212, 222 that preferably have an electrically conductive material, are made from such a material or are coated with it. The area 220 located proximally relative to the sleeve separator 260 is adjoined by a further proximal area 250 of the guide sleeve 200, which preferably has no further openings.

FIG. 2 shows a hose 300 which is guided in the lumen of the guide sleeve 200 and which, at its proximal end, is preferably connected to a fluid reservoir (not shown) in order to discharge an electrically conductive fluid 30, preferably a polymer-based NaCl gel, at its open end 310.

The several radial openings 211 and 221, respectively, which can be seen in particular in FIG. 3, can alternatively also be designed as a lattice structure, preferably a metal lattice structure, wherein preferably the guide sleeve 200 is designed as such a metal lattice structure in the area 210, 220.

An electrosurgical probe 100, with a rod-shaped probe body having a longitudinal direction LR, an outer surface and also a distal electrode 110 and a proximal electrode 120, is insertable into the guide sleeve 200 from the direction of the proximal end.

The electrosurgical probe 100 has a rounded distal tip 140 and, between the two electrodes 110, 120, an area 130 which electrically insulates the two electrodes 110, 120 from each other. In the proximal direction from the proximal electrode 120, a further proximal portion 150 of the electrosurgical probe 100 is formed. In the area 130 between the two electrodes 110, 120, the electrosurgical probe 100 has an expandable, electrically insulating probe separator 160, which is shown in the expanded state in FIGS. 4 and 5. The probe separator 160 is likewise designed as an expandable balloon, and the maximum diameter of the probe separator 160 in the expanded state is preferably adapted to the diameter of the guide sleeve 200 in the area 230 in such a way that the lumen of the guide sleeve 200 is filled completely in cross section by the probe separator 160, and it is thus possible to ensure a separation of the lumen of the guide sleeve 200 into two areas.

The sleeve separator 260 and the probe separator 160 can preferably both be converted from the contracted state to the expanded state shown, and back again, by in each case an actuation mechanism (not shown), preferably from the distal end of the electrosurgery arrangement 10.

The use of the embodiments of the electrosurgery arrangement 10 shown in the figures is preferably as follows. First, the guide sleeve 200, with its distal closed end 240 to the front, is inserted into a body lumen 20, for example a bronchus. When the guide sleeve 200 is located in the area of a target location, for example a tumor, the sleeve separator 260 is expanded. The body lumen 20 is then separated into two separate areas, which are electrically insulated from each other by the sleeve separator 260. Moreover, the sleeve separator 260 can also be used to fix the guide sleeve 200 at the exact position at the target location.

An electrically conductive fluid 30, for example a polymer-based NaCl gel, is introduced into the lumen of the guide sleeve 200. FIG. 2 shows, for this purpose, a hose 300 from the open distal end 310 of which a gel 30 emerges and fills the lumen of the guide sleeve 200. By way of the distal and proximal openings 211, 221 in the areas 210, 220 of the guide sleeve 200, the gel 30 leaves the lumen of the guide sleeve 200 and enters the body lumen 20 and fills the two areas located proximally and distally relative to the sleeve separator 260. In order to produce the contact made between the gel 30 and the wall of the body lumen 20, as shown in FIG. 3, sufficient gel has to be introduced into the body lumen 20 via the lumen of the guide sleeve and through the openings 211, 221.

When sufficient gel has been introduced, the hose 300 is removed from the lumen of the guide sleeve 200, and, from the direction of the proximal end of the guide sleeve 200, the electrosurgical probe 100, with its distal end 140 to the front, is inserted into the lumen of the guide sleeve 200. The electrosurgical probe 100 is preferably inserted into the lumen of the guide sleeve 200 until the distal end 140 of the guide sleeve abuts the closed distal end 240 of the guide sleeve. In this way, it is possible to ensure that the electrosurgical probe 100 can be positioned in a reproducible manner inside the guide sleeve 200. The guide sleeve 200 and the electrosurgical probe 100 are preferably adapted to each other in such a way that in particular the distances and extents in the longitudinal direction LR of the electrodes 110, 120 and of the areas 210 and 220 of the guide sleeve 200, in which the openings 211, 221 are located, come to lie in the same portions in the longitudinal direction LR.

In order also to produce two electrically insulated areas inside the gel-filled lumen of the guide sleeve 200, and moreover, if appropriate, also to fix the electrosurgical probe 100 inside the lumen of the guide sleeve 200, the probe separator 160 is brought from the contracted state to the expanded state shown in FIGS. 4 and 5. In the state of the electrosurgery arrangement 10, shown in FIG. 4, and of the areas filled with gel 30 and located proximally and distally relative to the sleeve separator 260 and also to the probe separator 160, an RF voltage can be applied to the electrodes 110, 120, which RF voltage is carried across the gel-filled areas of the lumen of the guide sleeve 200 and of the body lumen 20 via the interposed tissue, the latter being damaged by this current flow.

If, during the treatment, the gel distribution becomes uneven or is insufficient and, as shown in FIG. 5, an area 40 of the body lumen 20 is no longer sufficiently filled with gel, then the probe separator 160 can be contracted and the probe 100 removed from the lumen of the guide sleeve 200 in order to meter in new gel 30, as a result of which the situation shown in FIG. 2 arises (again). As soon as sufficient gel 30 has been metered in and the situation shown in FIG. 3 has been established (again), the electrosurgical probe 100 can be inserted again into the lumen of the guide sleeve 200 and, as is shown in FIG. 4, the probe separator 160 can be expanded again. By the distal end 140 of the electrosurgical probe 100 abutting against the distal end 240 of the guide sleeve 200, the exact, renewed positioning of the electrosurgical probe 100 is ensured.

### LIST OF REFERENCE SIGNS

1 bronchus
2 target location
3 electrosurgical probe
4 proximal electrode
5 distal electrode
6 electrically insulating balloon
7 gel
10 electrosurgery arrangement
20 body lumen
30 NaCl gel
40 area not adequately filled with gel
100 electrosurgical probe
110 distal electrode
120 proximal electrode
130 area between distal electrode and proximal electrode
140 distal end of the electrosurgical probe
150 proximal portion of the electrosurgical probe
160 probe separator
200 guide sleeve
210 area with radial openings (distal relative to the sleeve separator)
211 radial openings
212 surface between the radial openings
220 area with radial openings (proximal relative to the sleeve separator)
221 radial openings
222 surface between the radial openings
230 area between the radial openings
240 distal end of the guide sleeve
250 proximal area of the guide sleeve
260 sleeve separator
300 hose
310 distal end of the hose

1. An electrosurgery arrangement comprising
an electrosurgical probe with a rod-shaped probe body, an outer surface and also a distal electrode and a proximal electrode, wherein the distal electrode and the proximal electrode each form an electrically conductive portion of the outer surface and are electrically insulated from each other, and wherein an expandable, electrically insulating probe separator is arranged between the distal electrode and the proximal electrode; and
a guide sleeve with a lumen into which the electrosurgical probe is removably inserted, and with an expandable, electrically insulating sleeve separator, wherein the guide sleeve has at least one radial opening both proximally and distally relative to the sleeve separator.

2. The electrosurgery arrangement as claimed in claim 1, wherein the distal end of the guide sleeve is closed.

3. The electrosurgery arrangement as claimed in claim 1, wherein, with the electrosurgical probe inserted into the lumen as far as the distal end of the guide sleeve, the distal electrode is arranged inside the lumen in the area of the opening arranged distally relative to the sleeve separator, and the proximal electrode is arranged inside the lumen in the area of the opening arranged proximally relative to the sleeve separator.

4. The electrosurgery arrangement as claimed in claim 1, wherein the guide sleeve has several radial openings both proximally and distally relative to the sleeve separator, wherein the several radial openings are distributed in the circumferential direction of the guide sleeve.

5. The electrosurgery arrangement as claimed in claim 1, wherein
electrically conductive surfaces connected to each other are formed between the openings arranged proximally relative to the sleeve separator, and/or
electrically conductive surfaces connected to each other are formed between the openings arranged distally relative to the sleeve separator.

6. The electrosurgery arrangement as claimed in claim 1, wherein the guide sleeve, in the area of the openings arranged proximally relative to the sleeve separator and/or in the area of the openings arranged distally relative to the sleeve separator, is made from an electrically conductive material or has an electrically conductive material.

7. The electrosurgery arrangement as claimed in claim 1, wherein the openings arranged proximally relative to the sleeve separator and/or the openings arranged distally relative to the sleeve separator are designed as a lattice structure.

8. The electrosurgery arrangement as claimed in claim 1, wherein the guide sleeve has a lattice structure in the area of the openings arranged proximally relative to the sleeve separator and/or in the area of the openings arranged distally relative to the sleeve separator.

9. The electrosurgery arrangement as claimed in claim 1, wherein the lattice structure is made from an electrically conductive material or has an electrically conductive material.

10. The electrosurgery arrangement as claimed in claim 1, wherein the edge of the openings arranged proximally relative to the sleeve separator and/or of the openings arranged distally relative to the sleeve separator is made completely or partially from an electrically conductive material or has an electrically conductive material.

11. The electrosurgery arrangement as claimed in claim 1, wherein the expandable, electrically insulating sleeve separator and/or the expandable, electrically insulating probe separator are/is designed as an expandable balloon or as a deployable member.

12. A guide sleeve with a lumen into which an electrosurgical probe is removably insertable, and with an expandable, electrically insulating sleeve separator, wherein the guide sleeve has at least one radial opening both proximally and distally relative to the sleeve separator.

13. A method for operating an electrosurgery arrangement as claimed in claim 1, comprising the following steps:
inserting a guide sleeve with a lumen, into which an electrosurgical probe is removably insertable, and with an expandable, electrically insulating sleeve separator, wherein the guide sleeve has at least one radial opening both proximally and distally relative to the sleeve separator, into a body lumen as far as a target location,
expanding the sleeve separator,
introducing electrically conductive fluid into the lumen of the guide sleeve,
inserting an electrosurgical probe with a rod-shaped probe body, an outer surface and also a distal electrode and a proximal electrode, wherein the distal electrode and the proximal electrode each form an electrically conductive portion of the outer surface and are electrically insulated from each other, and wherein an expandable, electrically insulating probe separator is arranged between the distal electrode and the proximal electrode, into the lumen of the guide sleeve,
expanding the probe separator,
applying a bipolar RF voltage plied to the electrodes.

14. The method as claimed in claim 13, wherein the steps of:
removing the electrosurgical probe from the lumen of the guide sleeve,
introducing electrically conductive fluid into the lumen of the guide sleeve again,
inserting the electrosurgical probe into the lumen of the guide sleeve again,
expanding the probe separator again,
applying a bipolar RF voltage to the electrodes again.

15. The electrosurgical probe with a rod-shaped probe body, an outer surface and also a distal electrode and a proximal electrode, wherein the distal electrode and the proximal electrode each form an electrically conductive portion of the outer surface and are electrically insulated from each other, and wherein an expandable, electrically insulating probe separator is arranged between the distal electrode and the proximal electrode, and/or a guide sleeve with a lumen into which an electrosurgical probe is removably insertable, and with an expandable, electrically insulating sleeve separator, wherein the guide sleeve has at least one radial opening both proximally and distally relative to the sleeve separator, is used in an electrosurgery arrangement as claimed in claim 1, for operating an electrosurgery arrangement.
